(19) United States
(12) Patent Application Publication
(10) Pub. No.: US 2010/0067713 A1
(43) Pub. Date: Mar. 18, 2010

(54) METHOD FOR HEARING PROTECTING AND HEARING PROTECTION SYSTEM

(75) Inventors: Peter Boschung, Schmitten (CH); Martin Luetzen, Murten (CH); Erdal Karamuk, Meilan (CH); Gerard Van Oerle, Uster (CH); Hannes Oberdanner, Wolfhausen (CH); Hans Hessel, Benglen (CH)

Correspondence Address: PEARNE & GORDON LLP, 1801 EAST 9TH STREET, SUITE 1200, CLEVELAND, OH, 44114-3108, US

(73) Assignee: PHONAK AG, Staefa (CH)

(21) Appl. No.: 12/523,782

(22) PCT Filed: Jan. 30, 2007
(86) PCT No.: PCT/EP2007/050900

§ 371 Date: Oct. 15, 2009

(51) Int. Cl. A61F 11/06

(52) U.S. Cl. 381/72

(57) ABSTRACT

There is proposed a method and system for hearing protecting individuals, especially musicians. Thereby, such an individual is equipped with a hearing device (3, 5), the attenuation of acoustical signals impinging on such hearing device to signals transmitted towards the ear drum (8) is adjustable. There is provided a remote control unit (9), the output thereof being operationally connected to an attenuation control input (C₃) of the device (3). The remote control unit (9) is thereby operated differently than by the hands of the individual (1) which are frequently busy.

The present invention departs from hearing protection needs of growing importance for musicians. Thereby, it is important to note that whenever hearing protection needs for musicians arise, they have busy hands, i.e. they are at least intermittently practicing their proper instruments.

In spite of the fact that the present invention has been initiated by the addressed hearing protection needs of musicians, equal or at least very similar needs may be encountered for other individuals exposed to acoustical sceneries similar to sceneries experienced by musicians and which have busy hands too when exposed to such acoustical sceneries, so that the respective inventive solutions may also be applied for such individuals.

Further technical solutions as of the present invention initiated by accurate analysis of musicians' needs are considered of broader applicability than just for hearing protection devices, namely for hearing devices generically at which one operating mode is hearing protection.

We understand under the generic term of “hearing device” a device which is worn adjacent to or in an individual's ear with the object to improve an individual's acoustical perception. Such improvement may well be barring acoustical signals from being perceived in the sense of hearing protection for the individual user. If the hearing device is tailored so as to improve the perception of a hearing-impaired individual towards hearing perception of a standard individual, then we speak of a hearing aid device.

With respect to the application area, a hearing device may be applied at least with a part thereof behind the ear, may be applied in the ear or even completely in the ear canal, or may at least in part be implanted.

Musicians and especially musicians being members of performing groups as of brass bands, jazz bands, pop bands, symphonic orchestras, are often subjected to high sound pressure loads. This leads to the fact, as today recognized, that more and more frequency such individuals suffer from hearing damages. This is additionally amplified by the fact that today's audiences are becoming more and more desensitized with respect to acoustical volume perception, which leads to the tendency that orchestras perform increasingly loud. The social and economic damages which are caused by entire groups of population being hearing impaired already at relatively young ages are tremendous.

It is thus a starting point of the present invention to provide a hearing protection method and system which are suited to remedy the addressed problem.

When we specifically speak of “hearing protection” we address dampening sound pressure level from the acoustical surrounding of an individual with respect to transmission towards individual's ear drum. We speak throughout the present disclosure when addressing such dampening from “attenuation”. It has to be noted that hearing diseases which result from being exposed to respective high power acoustical signals for respective exposure times are, physiologically, mostly of mechanical nature, i.e. overload results at the inner ear structures caused by too high sound pressure.

Hearing protection of musicians poses specific problems which are normally not encountered in daily hearing protection appliances. Hearing protection for musicians should be dynamically controllable to different attenuation levels. E.g. during performing in a large symphony orchestra one addressed musician should be protected from acoustical overload at some specific times of performing, whereas at other times, e.g. when accompanying another musician in pianissimo, the addressed attenuation should be minimal.

As will be addressed, the basis for the method according to the present invention or to realize the system according to the present invention are hearing devices which have dynamically controllable attenuation between acoustical signals input from the acoustical surrounding to the device and an output signal towards individual's ear drum. Hearing devices which have such dynamically controllable attenuation are known. E.g. hearing aid devices are known which are cooperating with remote controls, allowing the individual hearing impaired person to adjust the addressed attenuation by hand.

Clearly, it is not practicable to provide for each individual musician, e.g. in a large symphony orchestra, a sophisticated hearing aid device just for hearing protection. Therefore, less sophisticated hearing devices, which are less costly than sophisticated hearing aid devices have been proposed in a co-pending patent application of the same applicant as for the present application which is, as not having been published yet, attached to the present disclosure as an Appendix.

Most generically and in the frame of the present invention we depart from the fact that hearing devices having dynamically controllable attenuation are known. They will therefore not be described in detail. It must nevertheless be stated that whenever, for other reasons than just for hearing protection, an individual, e.g. the addressed musician, has a more sophisticated hearing device with controllable attenuation, such sophisticated hearing device may be used to realize the method according to the present invention or may be incorporated into the system according to the invention. On the other hand less complex hearing devices just for hearing protection purposes may as well be applied to realize the addressed method and system, devices as e.g. disclosed in the addressed Appendix.

The present invention provides for a method for hearing protecting at least one individual having busy hands, which comprises applying to the individual a hearing device to at least one ear and controlling the attenuation from acoustical surrounding of the individual to an output of the at least one hearing device towards individual's ear drum, by a remote control which is operated differently than by individual's hands.

Thus, under a generic aspect of the method according to the present invention the attenuation at least one, most commonly at two hearing devices as worn by the individual is controlled without involving the hands of the addressed individual, so that e.g. a musician is not encumbered by attenuation control during practicing his instrument. It is to be noted that some musicians prefer to wear only one hearing protection device.

In one embodiment of the addressed method the control of attenuation comprises performing such control operated by the addressed individual's foot. Thereby, similarly as e.g. for a dictation machine, the individual may control the instantaneously prevailing attenuation by operating a foot pedal.

Normally, completely pushing down a foot-operable member by the individual will establish highest possible attenuation and thus the most protected mode. Nevertheless, it might also be, in some cases, that such complete push-down operation establishes the normal, relatively low-attenuation unprotected mode, and the individual switches by foot release into a more protected, higher attenuation mode.

According to a further embodiment of the addressed method, foot operation does not only control a binary level—on/off—attenuation, but does control a variable level of attenuation. Thus, the individual may e.g. by means of a foot pedal steadily increase or decrease momentarily prevailing attenuation.

In a further embodiment there is provided at least one further control of the addressed attenuation which is superimposed to the control operated by individual's foot. An example: Whenever e.g. in a large symphony orchestra each individual musician controls by foot pedal the attenuation of his proper hearing device, there might arise the situation where, due to a pianissimo passage for the entire orchestra, it would be detrimental when some of the individuals would forget to individually reset a previously high attenuation to lowest values and would erroneously play too loud. Therefore, an additional control is provided by which e.g. for all musicians of the orchestra, attenuation is minimized by a centralized control.

In a further embodiment, the addressed at least one further control of attenuation is operated by at least one of individual's movement and of a signal generated by a predetermined source.

It is known from hearing protection attempts for musicians that increased attenuation is frequently not estimated by the musicians during passages where they are playing themselves. Thus and under the present method according to the just addressed embodiment, individual's movement is detected so as to operate the addressed at least one further control, e.g. to reset a high attenuation back during playing movement. In another variant which may be used to the same target a signal generated by a predetermined source operates the addressed further control. E.g. the specific instrument of a musician may be selected as the addressed predetermined source and acoustical signals or mechanical vibration may be sensed to determine when the musician starts playing so as to reduce the high attenuation.

Clearly, such a signal generated by a predetermined source may also be an acoustical signal as generated by a performing group of musicians.

In most cases of such further control superimposed upon foot operated control, the further control reduces the individual's attenuation.

Still in a further embodiment of the method according to the present invention a group of individuals is hearing protected. Thereby, the individuals of the addressed group are each provided with a hearing device as was addressed and, respectively, at the addressed hearing devices the attenuations are controlled.

In this embodiment the attenuations at all the individuals of the addressed group are centrally controlled by one individual of the group. E.g. one member of an instrument group, as of the first violins, controls for all the members of the addressed group the respective individual attenuations. As was addressed, at least one further control, which is superimposed to such central group control, may again be individual as e.g. individually foot-operated, so that, within such group, by such control superposition individual attenuation settings may be additionally realized.

In a further embodiment of the method according to the generic concept of the present invention the control is operated automatically in dependency of characteristics of the individual's surrounding.

In one embodiment individual's surrounding is at least one of individual's acoustical and of individual's optical surrounding. Thus, the acoustical surrounding of the individual may be monitored and analyzed, whereby the attenuation at the at least one hearing device of the individual is varied in dependency of the analysis result. Further, e.g. in an opera house, the optical surrounding of the individual as with respect to light intensity and/or light color may be monitored and analyzed and individual's attenuation varied in dependency of the result of such analysis. Such an approach is practicable if there exists some correlation between performance intensity of an orchestra and optical effects adjacent the orchestra.

Still in a further embodiment the attenuation is varied in dependency of the addressed characteristics, such attenuation is not just switched on and off in the addressed dependency.

In one embodiment the addressed control is operated automatically in dependency of at least one of overall sound pressure level, of its time derivative, of its definite integral over a predetermined time span, of any derived values from sound pressure, time derivative thereof, definite time integral. All these values may thereby be considered over the entire hearable frequency spectrum or in selected, predetermined frequency bands within this spectrum. Thereby, such characteristic values are monitored over the hearable frequency band and as momentarily prevailing. If such characteristic value or values increase over predetermined threshold levels, as an example, the attenuation at the hearing device is increased. As addressed, additionally or alternatively, such characteristic values are monitored in specific frequency bands. Accordingly when such values rise above respective predetermined threshold values the overall or frequency band-specific attenuation is adjusted. Still as a further possibility there is monitored the increase rate of such values overall or in specific frequency bands, accordingly leading to increasing the overall attenuation or frequency band-specific attenuations.

In a further embodiment of operating automatically the control of attenuation there is superimposed to such control at least one further control. This further control may be an individual control as by foot operation or may be a control operated by individual movement and/or a signal generated by a predetermined source as was already addressed above.

In a further embodiment such at least one further control reduces the prevailing attenuation.

Still in a further embodiment of the variant performing automatically the individual attenuation control, a group of individuals is hearing protected, whereby a hearing device is applied to each ear of the individuals of the addressed group and there is performed at the respective hearing devices of each individual of the group the addressed automatic controlling, operated in dependency of the addressed surrounding sensed adjacent the one of the group members.

Still in a further embodiment and departing from the method according to the present invention under its generic aspect, the addressed control comprises performing such control operated by a further individual. Thereby, just as an example, in a symphony orchestra there might be a specific operator in charge of controlling individual's hearing device attenuation and/or of controlling such attenuation at the hearing devices of groups of musicians or of the entire orchestra.

In one embodiment there is again provided at least one further control which is superimposed on the addressed control which is operated by the further individual. Thereby, such a further control comprises in one embodiment a control which is operated by the one hearing protected individual. Thus, as an example, attenuation control at the addressed individual may be operated by a further individual and an additional further control of such attenuation is superimposed e.g. operated by the foot of the addressed individual.

Still in a further embodiment the further control is operated by at least one of individual's movement and of a signal generated by a predetermined source as was exemplified above.

In one embodiment the addressed further control reduces attenuation.

Whenever such control operated by a further individual is to be applied for protecting a group of individuals, such individuals are provided with a hearing device and there is performed at this hearing device the addressed control by the further individual.

In a further embodiment the addressed control is performed via a wireless communication link, whereby additionally, there may be provided wire-bound control. Thus, e.g. a more centralized attenuation control for a group of individuals may be realized via wireless links, whereas superimposed individual control e.g. foot-operable is realized via wire-bound links.

As was already addressed above, the method according to the present invention is especially suited for hearing protecting musicians.

According to the present invention, there is further provided a hearing protection system for at least one individual which comprises at least a hearing device for the individual, having a control input which is operationally connected to a controllable attenuation unit of the hearing device, which latter controls attenuation between an acoustical input to the hearing device and an output towards the ear drum of the individual. There is further provided a remote control unit with an input and with an output, whereby the output is operationally connected to the control input of the hearing device. The input of the remote control unit is a foot-operated member.

In one embodiment the addressed member is movable into different positions and the control unit generates different signals to the control input as a function of such position. Thus, the foot-operated member, as e.g. a pedal, may be pushed more or less down, which leads to generating varying signals, thereby controlling, via the addressed control input, an attenuation of varying level.

Still in a further embodiment of the addressed system there is provided at least one further control unit which has a further output being operationally connected to the addressed control input or, more generically, to a control input of the controllable attenuation unit. This allows superimposing different controls to the controllable attenuation unit.

Still in a further embodiment the just addressed further control unit comprises at least one of a movement detector and of a mechanical/electrical converter. Such mechanical/electrical converter may e.g. be a microphone, e.g. applied adjacent to an individual's instrument or a vibration detector placed on such instrument. The addressed movement detector may e.g. be an infrared movement detector placed nearby the instrument of the individual, so that there is detected whenever the addressed individual starts playing, allowing to adjust the attenuation to a level suited to such a situation.

In a further embodiment the addressed system comprises such a hearing device respectively for more than one individual and operationally connected to the addressed remote control unit which is foot-operated.

The present invention further provides for a hearing protection system for at least one individual, which comprises at least a hearing device for the individual. The at least one hearing device has a control input which is operationally connected to a controllable attenuation unit of the hearing device. The controllable attenuation unit controls attenuation between an acoustical input to said hearing device and an output towards individual's ear drum. There is further provided a remote control unit with an input and with an output. The output of the remote control unit is operationally connected to the control input of the hearing device and the input of the remote control unit is operationally connected to at least one of a mechanical/electrical converter and of an optoelectric converter.

By means of the addressed mechanical/electrical converter as of a microphone arrangement the momentarily prevailing acoustical surrounding may be monitored and the attenuation control is performed in dependency of such monitoring. By means of the addressed optoelectric converter e.g. in an opera house there might be additionally or alternatively controlled the attenuation in dependency of prevailing illumination.

Still in a further embodiment there is provided a signal analyzing unit which is interconnected between the converter and the controllable attenuation unit. By such signal analyzing unit the prevailing electric output signal of the converter is analyzed and, depending on the analyzing result, the attenuation at individual's hearing device is adjusted.

In a further embodiment there is provided at least one further control unit with a further output which is operationally connected to a control input of the controllable attenuation unit.

As was already addressed above and prevailing in context with the just addressed embodiment too, in a further embodiment such further control unit comprises at least one of a movement detector and of a further mechanical/electrical converter.

Still in a further embodiment a hearing device is respectively provided for more than one individual, thereby allowing to centrally control the attenuation at such hearing devices of a group of individuals.

The present invention further proposes a hearing protection system for at least one individual which comprises at least a hearing device for the individual. The at least one hearing device has a control input which is operationally connected to a controllable attenuation unit of the hearing device. The controllable attenuation unit controls attenuation between an acoustical input to the hearing device and an output towards individual's ear drum. There is further provided a remote control unit with an input and with an output. The output of the remote control unit is operationally connected to the control input of the hearing device and the input of the remote control unit is operationally connected to a man/machine input device which is not accessible by the addressed individual.

Thereby, the attenuation at the hearing device of the individual is remotely controlled by a further individual operating the addressed man/machine input device. Such an embodiment may be applied e.g. there where specific musicians shall not be bothered with respective attenuation control. Departing from the just addressed embodiment there is provided, in a further embodiment, again at least one further control unit with a further output which is operationally connected to a control input of the controllable attenuation unit. Advantages and possibilities which are reached by such at least one further control unit are apparent to the skilled artisan from the above disclosure and need no additional explanations.

As was already addressed in context with other embodiments of the system according to the present invention here too there is provided in one further embodiment the addressed at least one further control unit having at least one of a movement detector and of a mechanical/electrical converter.

Still in a further embodiment the system comprises the addressed hearing device respectively for more than one individual. Thereby, attenuation control is performed via the man/machine input device for a group of individuals centrally but not necessarily equally.

Still in a further embodiment which is valid for all embodiments of the system according to the present invention the output of the remote control unit is operationally connected to the control input via a wireless link. Thereby, an output of at least one further control unit as was addressed may nevertheless be operationally connected to the addressed input of the controllable attenuation unit by a wire-bound link, e.g. between a foot control unit and the hearing device.

The system according to the present invention is especially tailored for musicians.

The skilled artisan having read the disclosure up to now recognizes a huge number of different realization forms for the method and for the system according to the present invention. In spite of this, the invention shall now be even more amply disclosed by referring to specific examples which now shall be described with the help of figures.

The figures show:

FIG. 1: In a schematic representation, the generic embodiment of the method and system according to the present invention;

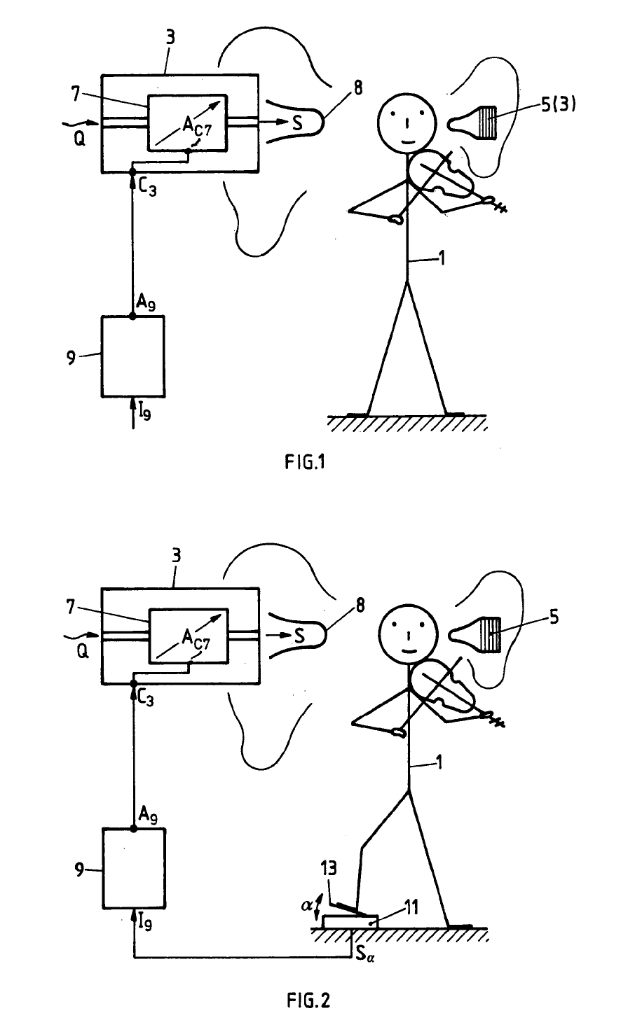

FIG. 2 departing from the embodiment of FIG. 1, an embodiment of the method and system according to the present invention by which the involved individual adjusts the amplification at the hearing device worn by himself;

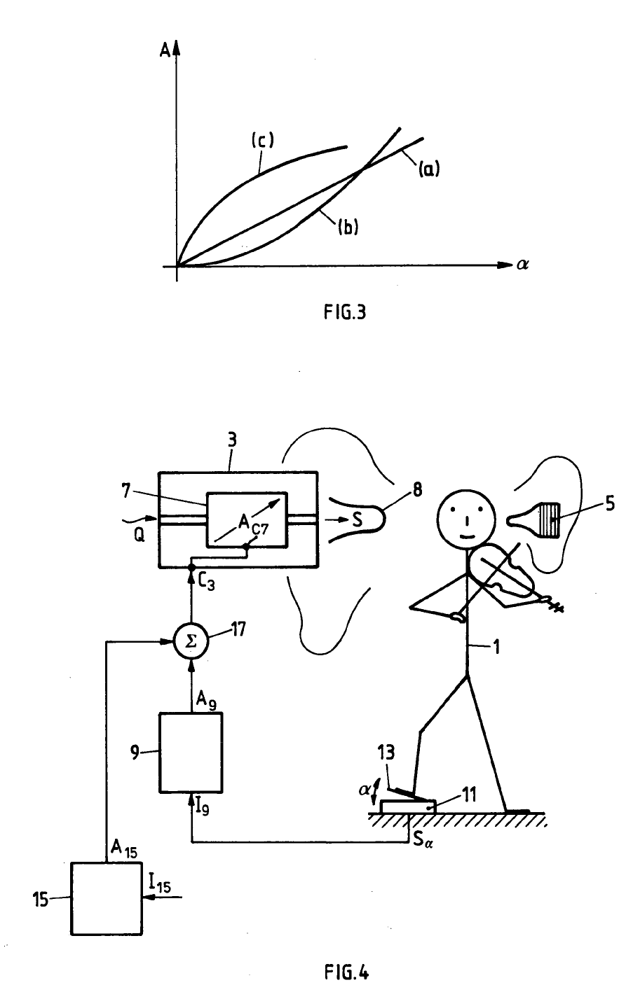

FIG. 3 three examples of amplification vs. operation characteristics in the embodiment of FIG. 2;

FIG. 4 departing from the embodiment according to FIG. 2, a further embodiment with superimposed further control of attenuation;

FIG. 5 departing from an embodiment according to FIG. 4, a still further embodiment of the method and system according to the present invention with semi-automatic individual adjustment of the attenuation;

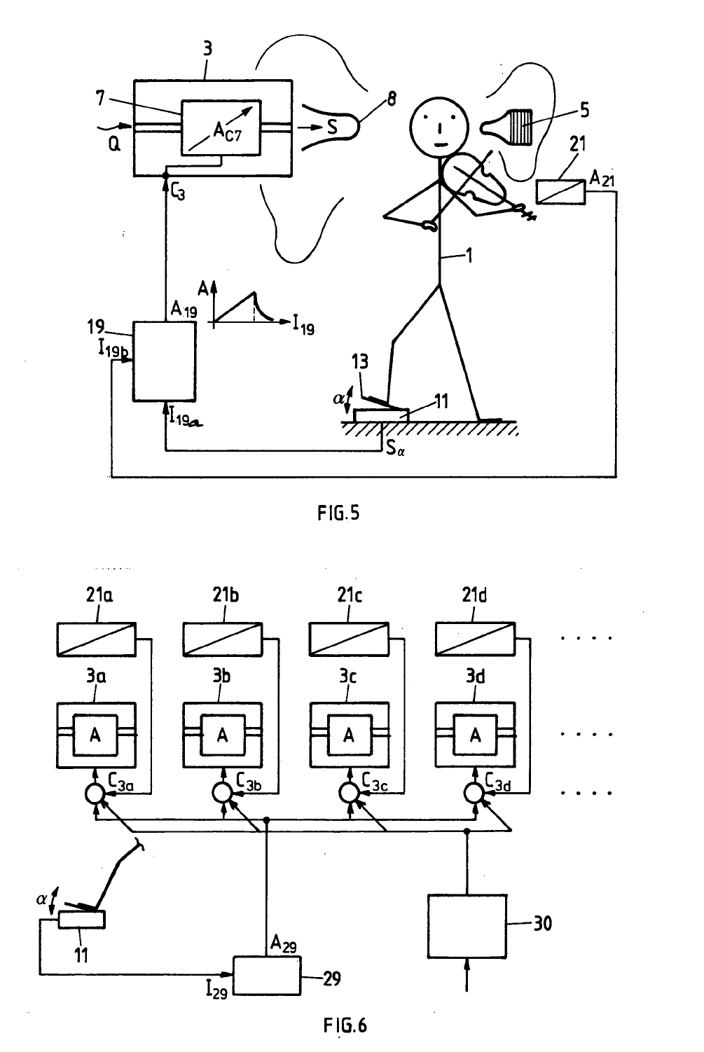

FIG. 6 in a schematic representation, centralized attenuation control for a group of individuals operated by a single individual, which is member of the group and/or attenuation control of the entire group by a central control not operated by a member of the group, and superimposed individual adjustment of the individual attenuations;

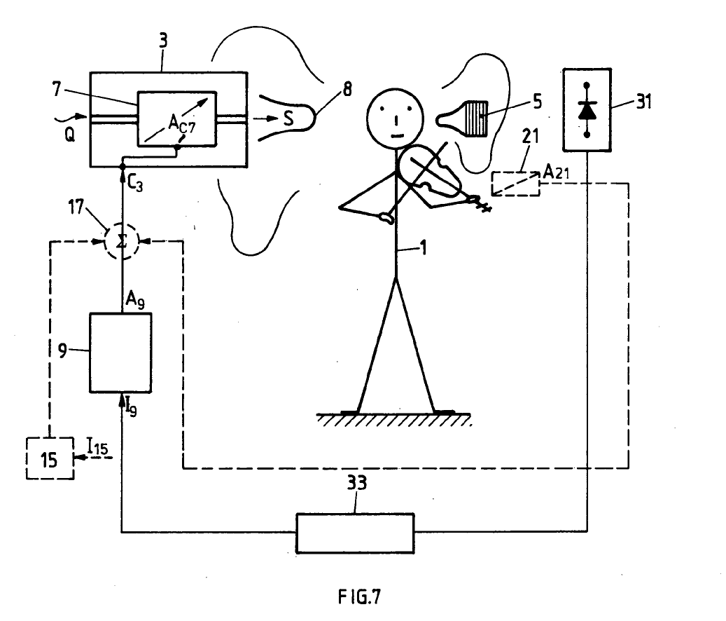

FIG. 7 a further embodiment according to which the attenuation at individual's hearing device is automatically controlled;

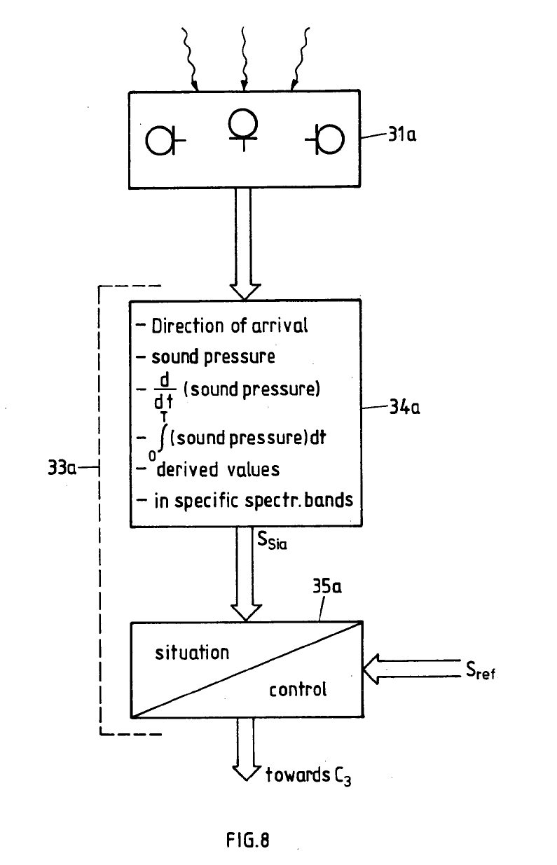

FIG. 8 a signal flow/functional block diagram of signal analysis as performed in one variant of the embodiment of FIG. 7;

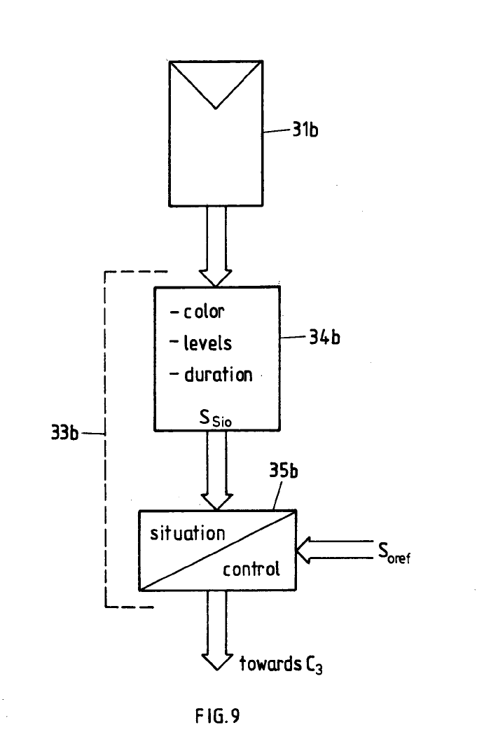

FIG. 9 in a representation in analogy to that of FIG. 8, a further variant of signal analysis;

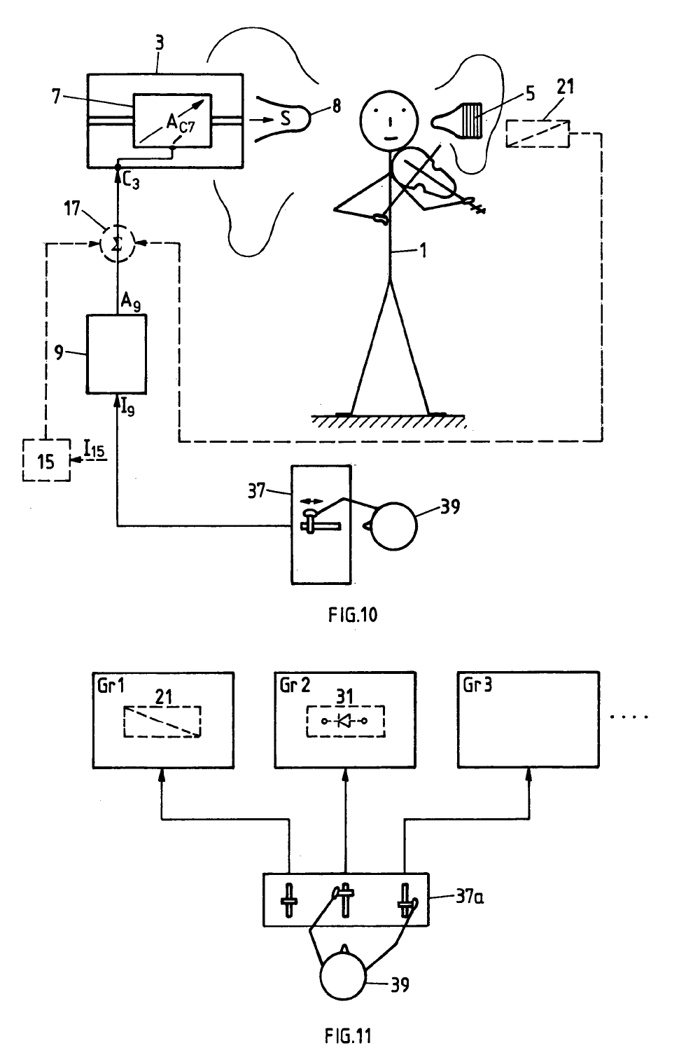

FIG. 10 departing from the method and system of the present invention as of FIG. 1, an embodiment in which the individual attenuation is controlled by a remote individual, and

FIG. 11 in a simplified diagrammatic form, attenuation control of different groups of individuals by an operator-individual.

In FIG. 1 there is schematically shown, under generic aspect, a hearing protection system for an individual performing the method according to the present invention. An individual 1 with busy hands, exemplified schematically by a musician playing his instrument, has applied, normally to both of his ears, exceptionally to one thereof, a hearing device 3 and 5. At least one of these normally two devices 3 and 5, according to FIG. 1 the device 3, has a controllable attenuation unit 7. As schematically shown in FIG. 1 the one hearing device 5—if applied at all—may just be a hearing protection device of passive type, in fact an ear plug, but will be in most practical cases a hearing device equal to the hearing device 3 with a controllable attenuation unit 7. The one or both hearing devices 3 may be realized by any known hearing device at which the attenuation between impinging acoustical signals from individual's surrounding Q to an output signal S towards the ear drum 9 of individual's ear, is controllable, be it commonly over the bearable frequency range and/or selectively in specific frequency bands, be it by varying the transfer function through the device, be it by varying a vent arrangement. Clearly, such a hearing device may be a sophisticated hearing device with complex digital signal processing as known from the art of hearing aid devices. Although such hearing aid devices often have a so-called “loudness limiter”, the loudness level to which the device output signal is limited is in practice often still too high to cope with ongoing overload of individual's ear as encountered in some groups of individual users, as in the generic group of musicians. The addressed device 3 as schematically shown in FIG. 1 may be a less complex device for actively controlled hearing protection, whereat the controllable attenuation unit 7 controls acoustical signal transmission e.g. from an input tubing open to the acoustical surrounding to an output tubing open towards individual's ear drum 9 which may be a vent channel too. Examples of such devices are disclosed in the already addressed co-pending patent application which is not published and therefore enclosed as an Appendix to the present disclosure.

Irrespective of the type of hearing device 3 the attenuation unit 7 has a control input C₇ which is operationally connected to an attenuation control input C₃ of device 3.

The system further comprises a remote control unit 9 which is operated differently than by the hands of individual 1. Operational connection of the control input C₃ to control output A₉ of remote control unit 9 is realized in wire-bound and/or in a wireless manner.

The remote control unit 9 has a control input I₉ to which a signal is applied which operates the remote control unit 9 to output a control signal to C₃ of the device 3 and, accordingly, to adjust attenuation A within attenuation control unit 7.

As already addressed, generating a control-operating signal to input I₉ of the control unit 9 is performed not involving the individual's hands, so that the individual 1 may continue to use his hands, be it at daily work or be it, specifically, as schematically shown in FIG. 1, for practicing as a musician his instrument.

In FIG. 2 a first embodiment of the method and system as has been explained with the help of FIG. 1 is shown in a representation analogous to that of FIG. 1. According to this embodiment the input I₉ of the remote control unit 9 is operationally connected to an operating member 11 which is operable by the foot 13 of the individual 1. Such member 11 may be realized, as shown, as a foot pedal, whereby such member 11 incorporates a respective converter for converting a mechanical movement of the pedal into an electrical signal applied to input I₉. In spite of the fact that by means of operable member 11 the attenuation might be binarily switched just from a low to a high level or vice versa, it is a good approach to provide attenuation control in dependency of the foot-operated position of member 11, according to FIG. 2, in dependency of the pedal angle α.

In FIG. 3 there is exemplified three different amplification—A—versus angle α characteristics which might be realized in the system according to FIG. 2. According to a first characteristic (a) the attenuation A at the attenuation control unit 7 is linearly varied with the pedal angle α. With the characteristic (b) the attenuation is progressively increasing as a function of the pedal angle α, and according to the characteristic (c) the attenuation is approaching an asymptotic maximum value as a function of increasing angle α. The conversion of an electric signal representing the angle α, S_{α} as of FIG. 2, to the respective control signal for adjusting the attenuation A according to a desired characteristic as exemplified in FIG. 3 is performed in the remote control unit 9. Nevertheless and if the device 3 is a more sophisticated device with digital signal processing ability, such conversion may also be realized by the respective processing abilities within the hearing device 3.

Departing from the representation of FIG. 2, FIG. 4 shows a further embodiment of the system and method according to the present invention. According to FIG. 4 there is superimposed to the control signal transmitted from the remote control unit 9 to the control input C₃ of the device 3, a further control signal from a further control unit 15 which is not operated by individual's hands too, but differently. The control unit 15 has a control signal output A₁₅ and an operating or setting input I₁₅. It is perfectly clear to the skilled artisan that superposition of the two control signals from output A₁₅ and from output A₉ may be realized in different manner. E.g. operational connection of A₉ to C₃ may be realized by a wire-bound link, whereas operational connection from A₁₅ to C₃ by a wireless link. Further, both addressed links may be realized wire-bound or both may be realized wirelessly. The superposition which is schematically shown at unit 17 of FIG. 4 may be realized at one of the remote control unit 9 and of the remote control unit 15 or at the hearing device 3, e.g. at control input C₇.

The further control unit 15 may be provided, as will be exemplified later, so as to control attenuation A simultaneously at hearing devices 3 worn by individuals of a group of individuals. Such a group may be an instrument group of an orchestra or a group of musicians similarly exposed to acoustical loading, as being seated in front of brass instruments. The further control 15 may further be used as exemplified in FIG. 5 to automatically control the attenuation A at individual's hearing device 3 in specific situations.

According to FIG. 5 and with an eye on the embodiments according to FIGS. 1 to 4, the remote control unit 19 performs superposition of the two controls as addressed in context with FIG. 4. Thereby and as schematically shown the operating signal from foot-operable member 11 is fed to input I₁₉ₐ and a further control-operating signal is fed to a second input I_{19b} of control-superposing remote control unit 19. The second input I_{19b} is operationally connected to an output A₂₁ of a converter unit 21 which comprises at least one of an optoelectric and of a mechanical/electrical converter. By means of the converter unit 21 the surrounding to which the individual 1 is exposed is monitored. The converter unit 21, as a mechanical/electrical converter, may be realized by a vibration sensor or a microphone applied to or nearby the instrument of a musician, thereby monitoring whether such instrument is active as a predetermined source of a signal.

Especially with an eye on musicians, it is known from practice that whenever a musician plays himself, he prefers to have installed a relatively low attenuation. Thus, whenever the addressed converter unit 21 generates an electric output signal which is indicative for the instrument being active, the attenuation A previously set to a higher level is automatically reduced. As schematically shown in FIG. 5 the musician which has increased the attenuation A by means of member 11, via input I₁₉ₐ of remote control unit 19, will then experience automatically a reduced attenuation A. Reduction is initiated and controlled by the converter unit 21 generating an output signal to I_{19b}, indicative for activity of musician's instrument.

Instead of providing a converter unit 21 as a mechanical/electrical converter or additionally thereto, there may be provided an optoelectric converter, e.g. an infrared movement detector, thereby monitoring movement of the musician, so as to detect active playing.

Departing from the embodiment as has been explained with the help of the FIGS. 2-5 the embodiment as shown in FIG. 6 provides for central attenuation control of more than one hearing device, each applied to individuals of a group, as of musicians. The attenuation of the devices is controlled by one individual's foot operation. Thereby, in the embodiment of FIG. 6, there is provided for each individual of the group a further control as has been exemplified in FIG. 5 so that individually and for specific situations, the centrally controlled attenuation may individually be varied. According to FIG. 6 more than one individual, i.e. individuals of a group, are each equipped with at least one hearing device 3a, 3b, 3c, 3d, etc. The respective individuals wearing the addressed at least one hearing device are not shown in FIG. 6 for clearness' sake. Nevertheless, the respective individuals shall be addressed with the same reference number 3a to 3d etc. as used for specifying their hearing devices. One of the individuals, e.g. individual 3a, is equipped with member 11 as has been described with the help of FIGS. 2-5. The output of a remote control unit 29 is operationally connected to each of the hearing devices 3a to 3d of the individuals of the group. Thus, whenever the one “master” individual actuates the foot-operable member 11 and with an eye on FIG. 3 the amplifications A of all the addressed hearing devices are equally or differently varied. Thus, in a first embodiment there is performed centralized amplification control of a group of individuals, especially of musicians. Clearly, if there is need, the attenuations in the group need not be equally adjusted.

As also shown in FIG. 6 and as a further embodiment there is provided at each individual and as has been explained with the help of FIG. 5 a converter unit 21a, 21b, etc., etc., by which an individual's specific situation is monitored. Such a situation for an individual musician is e.g. activity of playing. The respective output of the converters 21a, 21b, etc. are respectively superimposed to the central control by “master” individual 3a so as to provide for individually superimposed attenuation control. In spite of the fact that, according to FIG. 6, such superposition is shown within the operational connection between output A₂₉ of remote control unit 29 and the respective control inputs C₃ₐ to C_{3d} it is advisable to provide control superposition directly at the respective hearing devices 3a to 3d. Thereby, in one embodiment the output A₂₉ may be linked to the control inputs C₃ₐ to C_{3d} by respective wireless links and the respective converter units 21a to 21d may be linked to the respective control inputs C₃ₐ to C_{3d} by wire-bound links and/or by wireless links, which latter are individually paired to the individual converters 21 and hearing devices 3 e.g. frequency-selective. In this embodiment and as was already addressed all the individuals of the group are e.g. synchronously controlled centrally, e.g. to increase respective attenuations, whereby and with an eye on musicians, whenever one of the musicians of the group starts playing a relatively high attenuation is individually reduced via the individual converter unit 21.

As further shown in FIG. 6 there may be as in a further embodiment superimposed to all individual attenuation controls a still further control from a still further control unit 30, e.g. to take control upon all the attenuations of the group by a further individual, as by an individual in charge of attenuation control for a complete symphony orchestra. E.g. whenever such orchestra has to play pianissimo it might be advisable to overwrite all the group attenuation settings as well as all the individual attenuation settings by an orchestra-wide attenuation control on a low level.

In FIG. 7 and departing from the method and system as generically shown in FIG. 1 there is represented a further embodiment, whereat the attenuation A of the at least one hearing device 3 worn by the individual 1 is controlled operated automatically in dependency of characteristics of individual's surrounding. There is provided nearby the individual 1 a sensor arrangement 31 which acts as a converter, outputting an electrical signal which is indicative for surrounding characteristics which are monitored by the sensor arrangement 31. The output of the sensor arrangement 31 is operationally connected to input I₉ of remote control 9 so as to operate such control unit and to generate at its output A₉ a control signal wirelessly or wire-bound applied to control input C₃ of hearing device 3. Thereby, sensor arrangement 31 may be or may comprise a mechanical/electrical converter arrangement as of a microphone arrangement to monitor the acoustical surrounding of individual 1. Additionally or instead of such mechanical/electrical converter arrangement the sensor arrangement 31 may comprise an optoelectric converter so as to monitor ambient light in individuals's surrounding. There is further provided, e.g. interconnected between the output of the sensor arrangement 31 and the output A₉ of the remote control unit 9, a signal analyzing unit 33 which, in the representation of FIG. 7, is shown as a separate unit. Clearly, such signal analyzing unit may be incorporated in the remote control unit 9.

FIG. 8 schematically shows, as an example and by means of a signal flow/functional block diagram, a sensor arrangement 31a being realized as an arrangement of mechanical/electrical converters as of microphones. In a signal analyzer unit 33a, according to analyzing unit 33 tailored to analyze the acoustical surrounding of individual 1, in a first stage 34a and from signals provided from the sensor unit 31a at least one of direction of arrival of acoustical signals impinging on the arrangement 31a and of sound pressure of such time derivative of sound pressure and of definite integral of sound pressure over a predetermined time T and of values derived from sound pressure, time derivative thereof, definite integral thereof are evaluated. Such values are evaluated over the entire especially hearable frequency spectrum and/or in predetermined specific frequency bands within the addressed spectrum. The respective electric signals S_{Sia} specifying the momentarily prevailing acoustical situation to which the individual 1 is exposed are lead to a situation-to-control signal converter unit 35a. Therein, generically there is performed a comparison of the signals identifying to the desired extent the acoustical situation, S_{Si}, with pre-established reference signal S_{ref} and in dependency of the result or of the results of such comparing there is output at least one control signal which is operationally connected to the control input C₃ of device 3. Thereby, the attenuation A is, in one embodiment, not only binarily set to a low level or to a high level, but is adjusted on different levels in dependency of the addressed one or more than one comparison results. The attenuation is thereby adjusted over the hearable spectrum or in selected spectral bands within this spectrum.

In this embodiment the attenuation A of the hearing device 3 is automatically varied in dependency of the momentarily prevailing acoustical situation.

So as not to disturb the individual 1 and especially a musician with ongoing changes of attenuation, it might be advisable in this embodiment as well as in other embodiments to apply quantization of the attenuation, e.g. on five different levels, and/or to perform changes of attenuation level rather continuously than in steps.

In FIG. 9 there is shown in a representation in analogy to that of FIG. 8 operation of the sensor arrangement 31 tailored as an optoelectric converter 31b with a stage 34b tailored for optical signal evaluation and with a respective situation-to-control signal converter unit 35b. By means of the optoelectric converter arrangement 31b the illumination surrounding of the individual 1 is monitored and the output signal is applied to evaluation unit 34b as a part of analyzing unit 33b. The signal or signals output from converter 31b are analyzed e.g. with respect to color content, brightness, duration, etc. Much in analogy to the embodiment of FIG. 8, the analyzing results define for the situation—now the optical situation—experienced by the individual and are transmitted as signals S_{Sio} to the situation-to-control signal converter unit 35b. The situation-to-control signal converter unit 35b operates equally with respect to the optical situation identifying signals S_{Sio} as was explained with respect to unit 35a for acoustical situation identifying signals S_{Sia} in context with FIG. 8.

As is shown in FIG. 7 by dashed lines, also in this embodiment according to which attenuation control is automatically performed, there is provided in a further embodiment and much in analogy to the embodiment 4 at least one further control which is superimposed to the control which is provided and operated from the sensor arrangement 31. If the individual is a member of a group, the attenuation of the hearing devices of the group members having to be varied or set simultaneously as was explained in context with FIG. 6, such a further control may be operated centrally for the entire group. Alternatively or additionally to such centrally operated control and as further shown in FIG. 7 in dashed lines, in a further embodiment, there is provided the converter arrangement 21 as was explained in context with FIG. 5 or 6, by which e.g. time spans of active playing by the individual musician are detected and during such time spans, individually, the attenuation A is reduced. Still a further control which may be superimposed to the addressed automatic control by the sensor arrangement 31 may be an individual control e.g. by means of a foot-operated member 11 as was explained in context with the FIGS. 1 to 5, by means of which the individual may overwrite or influence the automatic attenuation control. With an eye on FIG. 7 and generically directed to superimposing different controls for the attenuation of individual's hearing device and under a further aspect, such different controls may be weighted with different priorities. Thus, e.g. a central group control as by control unit 15 in FIG. 7 may be of primary priority overriding automatic control as well as instrument monitoring controls as by converters 21.

Further and as was already addressed in context with FIG. 4 it has to be emphasized that control superposition as schematically represented by superposition unit 17 in FIG. 7 too is, in practice, realized within remote control unit 9 or within hearing device 3. This also depends from the fact by which kind of link, wire-bound or wireless, the different controls are communicated to the hearing device 3, as schematically shown to control input C₃ and finally to an attenuation control input of unit 7.

With an eye on FIG. 6 we have already addressed simultaneously varying the attenuation A of hearing devices 3a to 3d by a common control as of 30 or 29. Clearly, the automatic attenuation control as has been described in context with FIG. 7 may be applied for a central amplification control for a group of individuals with the respective hearing devices. As an example the additional control unit 30 as of FIG. 6 may be operated by a sensor arrangement 31 as of FIG. 7, whereby the acoustical and/or optical surrounding which prevails for the addressed group of individuals is sensed by such sensor arrangement 31.

Departing again from the generic principle of the method and system according to the present invention and as shown in FIG. 1, FIG. 10 shows a further embodiment. Here the amplification A is controlled by the remote control unit 9 operated by a setting unit 37 which is operated by an operator individual 39. As shown in dashed lines in FIG. 10 additional controls may be superimposed in further embodiments, as was already explained in context e.g. with the embodiment of FIG. 7. Still in a further embodiment and as schematically shown in FIG. 11 the operator 39 operates a setting unit 37a, whereat the control of attenuation is adjusted separately for respective groups Gr1, Gr2, etc. of individuals, as of musicians. Thereby, as shown in dashed lines in the block representing first group Gr1, one of the groups may further be equipped with the arrangements 21 as has been specifically described in context with FIG. 5, a second group, as Gr2, may be equipped with a group-specific automatic further control by means of sensor arrangement 31 as has been specifically described in context with FIG. 7, etc. Thus, the skilled artisan will recognize that the addressed amplification control for individuals and/or groups may be superimposed according to the specific needs in a large number of combinations. The embodiment as of FIG. 11 is e.g. suited for a large symphony orchestra, where an operator 39 is affordable so as to disburden the individual musician or groups of musicians from the task of properly and adequately adjusting the attenuation of the respective individuals and/or groups.

1. A method for hearing protecting at least one individual having busy hands comprising:
Applying to said individual a hearing device to at least one ear;
Controlling along said hearing device attenuation from acoustical surrounding of said individual to an output of said hearing device towards individual's ear drum by a remote control operated differently than by said individual's hands.

2. The method of claim 1, performing said control comprising performing said control operated by individual's foot.

3. The method of claim 2, comprising controlling a variable level of attenuation by individual's foot.

4. The method of claim 2 or 3, further comprising superimposing to said control operated by individual's foot at least one further control.

5. The method of claim 4, said at least one further control of said attenuation being operated by at least one of individual's movement and of a signal generated by a predetermined source.

6. The method of claim 4 or 5, said at least one further control reducing said attenuation.

7. The method of one of claims 2 to 6 for protecting a group of individuals including said one individual, comprising
applying to the other individuals of said group at least one hearing device;
controlling along said hearing devices of said individuals said attenuations respectively by said control.

8. The method of claim 1, performing said control comprising performing said control operated automatically in dependency of characteristics of individual's surrounding.

9. The method of claim 8, said surrounding being at least one of acoustical and of optical surrounding.

10. The method of one of claim 8 or 9 comprising varying said attenuation in said dependency.

11. The method of one of claims 8 to 10, said characteristics being one of overall sound pressure level, of its time derivative, of its definite integral over a predetermined time span, of any value derived from at least one of sound pressure, time derivative thereof, definite time integral thereof, of at least one of said sound pressure, said derivative, said integral, said derived values in specific frequency bands.

12. The method of claims 8 to 11, further comprising superimposing to said control operated automatically at least one further control.

13. The method of claim 12, said at least one further control comprising a control operated by said individual.

14. The method of claim 12 or 13, said at least one further control of said attenuation being operated by at least one of individual's movement and of a signal generated by a predetermined source.

15. The method of one of claims 12 to 14, said at least one further control reducing said attenuation.

16. The method of one of claims 8 to 15 for protecting a group of individuals, comprising
applying to the other individuals of said group a hearing device;
controlling along said hearing devices of said individuals said attenuations respectively by said control.

17. The method of claim 1, comprising performing said control operated by a further individual.

18. The method of claim 17, further comprising superimposing to said control operated by said further individual at least one further control.

19. The method of claim 18, said at least one further control comprising a control operated by said individual.

20. The method of claim 18 or 19, said further comprising a control operated by at least one of individual's movement and of a signal generated by a predetermined source.

21. The method of one of claims 18 to 20, said at least one further control reducing said attenuation.

22. The method of one of claims 17 to 21 for protecting a group of individuals, comprising
applying to the individuals of said group at least one hearing device;
controlling along said hearing devices of said individuals said attenuations respectively by said control.

23. The method of one of claims 1 to 22, performing said control comprising performing said control via a wireless communication link.

24. The method of one of claims 1 to 23 for protecting musicians.

25. A hearing protection system for at least one individual comprising
at least one hearing device for said individual, having a control input operationally connected to a controllable attenuation unit of said hearing device, controlling attenuation between an acoustical input to said hearing device and an output towards individual's ear drum;
a remote control unit with an input and with an output, said output being operationally connected to said control input, said input being a foot operated member.

26. The system of claim 25, said member being movable into different positions, said control unit generating different signals to said control input as a function of said positions.

27. The system of one of claim 25 or 26, comprising at least one further control unit with a further output operationally connected to said controllable attenuation unit.

28. The system of claim 27, said further control unit comprising at least one of a movement detector and of a mechanical/electrical converter.

29. The system of one of claims 25 to 28, comprising said hearing devices for more than one individual.

30. A hearing protection system for at least one individual comprising
at least one hearing device having a control input operationally connected to a controllable attenuation unit of said hearing device, controlling attenuation between an acoustical input to said hearing device and an output towards individual's ear drum;
a remote control unit with an input and with an output, said output being operationally connected to said control input, said input being operationally connected to at least one of a mechanical/electrical converter and of an optoelectric converter.

31. The system of claim 30, further comprising a signal analyzing unit interconnected between said converter and said controllable attenuation unit.

32. The system of one of claim 30 or 31, comprising at least one further control unit with a further output operationally connected to said controllable attenuation unit.

33. The system of claim 32, said further control unit comprising at least one of a movement detector and of a further mechanical/electrical converter.

34. The system of one of claims 30 to 33, comprising said hearing devices for more than one individual.

35. A hearing protection system for at least one individual comprising
at least one hearing device having a control input operationally connected to a controllable attenuation unit of said hearing device, controlling attenuation between an acoustical input to said hearing device and an output towards individual's ear drum;
a remote control unit with an input and with an output, said output being operationally connected to said control input, said input being operationally connected to a man/machine input device being not accessible by said individual.

36. The system of claim 34, comprising at least one further control unit with a further output operationally connected to said controllable attenuation unit.

37. The system of claim 36, said further control unit comprising at least one of a movement detector and of a mechanical/electrical converter.

38. The system of one of claims 35 to 37, comprising said hearing devices for more than one individual.

39. The system of one of claims 25 to 38, said output of said remote control unit being operationally connected to said control input via a wireless link.

40. The system of one of claims 25 to 39 for musicians.
